(19) United States
(12) Patent Application Publication
(10) Pub. No.: US 2009/0306425 A1
(43) Pub. Date: Dec. 10, 2009

(54) Production of Carboxylic Acids

(76) Inventors: Didier Bonnet, Lyon (FR); Romain Petroff Saint-Arroma, Paris (FR); Sebastien Righini, Lyon (FR); Tania Ireland, Lyon (FR); Jean-Pierre Simonato, Sassenage (FR)

Correspondence Address: BUCHANAN, INGERSOLL & ROONEY PC, POST OFFICE BOX 1404, ALEXANDRIA, VA, 22313-1404, US

(21) Appl. No.: 11/922,333

(22) PCT Filed: Jun. 9, 2006
(86) PCT No.: PCT/FR2006/001308

§ 371 Date: May 6, 2009

(30) Foreign Application Priority Data
Jun. 17, 2005 (FR) 05 06160

(51) Int. Cl. C07C 51/21

(52) U.S. Cl. 562/542; 562/400

(57) ABSTRACT

A process for the production of carboxylic acids by oxidation of a hydrocarbon by oxygen or a gas containing oxygen and notably to the oxidation of cyclohexane to give adipic acid; the subject process entails a stage of oxidation of the hydrocarbon and at least one stage for extracting the dicarboxylic acids formed from the reaction medium and optionally recycling the unconverted hydrocarbon with oxidation by-products, such as alcohols and ketones, and which also includes a stage of conversion, removal or extraction of the α,ω-hydroxycarboxylic compounds formed during the oxidation stage and converting these compounds into diacids.

The present invention relates to a process for the manufacture of carboxylic acids.

It relates more particularly to a process for the manufacture of carboxylic acids by oxidation of a hydrocarbon by oxygen or a gas comprising oxygen and more particularly still to the oxidation of cyclohexane to give adipic acid.

Adipic acid is an important chemical compound used in numerous fields. Thus, adipic acid can be used as additive in numerous products, both in the food field and in concretes. However, one of the most important uses is its application as a monomer in the manufacture of polymers, including polyurethanes and polyamides.

Several processes for the manufacture of adipic acid have been provided. One of the most important, used industrially on a large scale, consists in oxidizing cyclohexane by a gas comprising oxygen or by oxygen, in one or two stage(s), to give a cyclohexanol/cyclohexanone mixture. After extracting and purifying the cyclohexanol/cyclohexanone mixture, these compounds are oxidized, in particular to give adipic acid, by nitric acid.

However, this process exhibits a major disadvantage relating to the formation of nitrous vapour.

Numerous studies have been carried out to develop a process for the oxidation by oxygen or a gas comprising oxygen of hydrocarbons which makes it possible to directly obtain the carboxylic acids, mainly adipic acid.

These processes are disclosed in particular in Patents FR 2 761 984, FR 2 791 667, FR 2 765 930 and U.S. Pat. No. 5,294,739.

Generally, the reaction is carried out in a solvent medium, the solvent being a monocarboxylic acid, such as acetic acid. Other solvents have been provided, such as the carboxylic acids possessing a lipophilic nature disclosed in Patent FR 2 806 079.

Numerous patents have disclosed the operating conditions for this reaction, have described the various stages for extracting the acids formed, for purifying them and also for recycling the nonoxidized hydrocarbon, and have described the catalyst.

However, in this oxidation reaction, by-products are formed which can to a more or less significant extent reduce the yield of the process. Some of these, such as alcohols, react with the acids formed to give esters which have to be extracted from the reaction medium to prevent their accumulation or the production of undesirable impurities difficult to separate from the acids formed.

Other intermediate oxidation products, such as α,ω-hydroxycarboxylic compounds, are also troublesome if they are not removed from the reaction medium or converted. This is because these compounds are often difficult to separate from the diacids, making it difficult to obtain a pure acid exhibiting in particular the degree of purity required for use as monomer in the manufacture of polyamides.

It is important for the economics of the process and also for production of diacids with a high degree of purity to reduce the concentration of by-products in the reaction medium and in particular in the diacids recovered.

One of the aims of the present invention is to provide a process for the manufacture of diacids which makes it possible to remove, extract or convert the by-products resulting from the oxidation reaction.

To this end, the invention provides a process for the manufacture of dicarboxylic acids by oxidation of a cycloaliphatic hydrocarbon with molecular oxygen or a gas comprising molecular oxygen in the presence of a solvent.

According to the invention, the process comprises a stage of oxidation of the hydrocarbon and at least one stage for extracting the dicarboxylic acids formed from the reaction medium and optionally recycling the unconverted hydrocarbon with oxidation by-products, such as alcohols and ketones, which may be formed.

The process of the invention also comprises a stage of conversion, removal or extraction of the α,ω-hydroxycarboxylic compounds formed during the oxidation stage.

This stage of conversion, removal or extraction of the α,ω-hydroxycarboxylic compounds consists in subjecting the medium comprising these compounds to an oxidation in order to convert them to diacids. This oxidation reaction is optionally carried out in the presence of a catalyst comprising, as catalytically active component, a metal or metal compound chosen from the group consisting of Cu, Ag, Au, Mg, Ca, Sr, Ba, Zn, Cd, Hg, Al, Sc, In, Tl, Y, Ga, Ti, Zr, Hf, Ge, Sn, Pb, V, Nb, Ta, Cr, Mo, W, Mn, Tc, Re, Fe, Ru, Os, Co, Rh, Ir, Ni, Pd, Pt, lanthanides, such as Ce, and combinations of these, preferably precious metals, such as platinum, gold, silver, ruthenium, rhenium, palladium or their mixtures. Advantageously, this catalytically active metal or metal compound is deposited on, impregnated onto or grafted to a porous support, such as carbon black, alumina, zeolites, silica, graphite and more generally the supports used in the field of catalysis.

The preferred catalyst of the invention is in particular a catalyst comprising a platinum compound deposited on carbon black.

The reaction for the oxidation of the α,ω-hydroxycarboxylic compounds is advantageously carried out at a temperature of between 50 and 150° C.

The oxidizing agent suitable for this stage is advantageously molecular oxygen or a gas comprising molecular oxygen. It is also possible to use other oxidizing agents, such as aqueous hydrogen peroxide solution, ozone or nitric acid.

According to a first embodiment of the invention, the stage of conversion, removal or extraction of the hydroxycarboxylic compounds is carried out on the medium exiting from the oxidation reactor prior to the separation of the diacids formed and of the unreacted hydrocarbon, that is to say in the presence of the organic phase.

According to a second embodiment of the invention, the stage of conversion of the hydroxycarboxylic compounds is carried out on the medium comprising the diacids formed after extraction of the latter from the oxidation reaction medium or, after crystallization of the diacids, on the aqueous crystallization mother liquors, that is to say on a medium composed of an aqueous phase.

Thus, in the first embodiment of the invention, the homogeneous or heterogeneous oxidation catalyst is added to the reaction medium, either to the oxidation reactor after the end of the reaction for oxidation of the hydrocarbon or to one or more separate oxidation reactors to which the reaction medium is fed. In this embodiment, the catalyst used is advantageously a homogeneous metal catalyst or a mixture of homogeneous catalysts. The temperature condition is defined and is, for example, between 50 and 150° C.

The oxidizing agent is advantageously oxygen or a gas comprising oxygen, such as air, for example. In this case, the oxygen partial pressure is advantageously between 0.1 and 30 bar.

In the second embodiment of the invention, the oxidation of the α,ω-hydroxycarboxylic compounds is carried out in an aqueous medium, either in the absence of a catalyst or in the presence of a catalyst as defined below. Advantageously, the catalyst is a heterogeneous catalyst and the oxidizing agent is oxygen, a gas comprising oxygen, nitric acid, aqueous hydrogen peroxide solution or ozone, for example.

The process of the invention applies in particular to the oxidation of cyclohexane to produce adipic acid. It can also be applied to the oxidation of other hydrocarbons, such as cyclododecane.

The reaction for the oxidation of the hydrocarbon, for example cyclohexane, is generally carried out in the presence of a solvent. This solvent can be highly varied in nature, in so far as it cannot be oxidized under the reaction conditions. It can in particular be chosen from polar protic solvents and polar aprotic solvents. Mention may be made, as polar protic solvents, for example, of carboxylic acids having only primary or secondary hydrogen atoms, in particular aliphatic acids having from 2 to 9 carbon atoms, such as acetic acid, perfluoroalkanecarboxylic acids, such as trifluoroacetic acid, alcohols, such as tert-butanol, halogenated hydrocarbons, such as dichloromethane, or ketones, such as acetone. Mention may be made, as polar aprotic solvents, for example, of lower alkyl (=alkyl radical having from 1 to 4 carbon atoms) esters of carboxylic acids, in particular aliphatic carboxylic acids having from 2 to 9 carbon atoms or perfluoroalkanecarboxylic acids, tetramethylene sulphone (or sulfolane), acetonitrile or benzonitrile.

The solvent can also be chosen from carboxylic acids possessing a lipophilic nature.

The term “lipophilic acid compound suitable for the invention” is understood to mean aromatic, aliphatic, arylaliphatic or alkylaromatic acid compounds comprising at least 6 carbon atoms which can comprise several acid functional groups and which exhibit a low solubility in water, that is to say a solubility of less than 10% by weight at ambient temperature (10° C.-30° C.).

Mention may be made, as lipophilic organic compound, for example, of hexanoic, heptanoic, octanoic, 2-ethylhexanoic, nonanoic, decanoic, undecanoic, dodecanoic or stearic (octadecanoic) acids and their permethylated derivatives (complete substitution of the hydrogens of the methylene groups by the methyl group), 2-octadecylsuccinic acid, 3,5-di(tert-butyl)benzoic acid, 4-(tert-butyl)benzoic acid, 4-octylbenzoic acid, tert-butyl hydrogen orthophthalate, naphthenic or anthracenic acids substituted by alkyl groups, preferably of tert-butyl type, substituted derivatives of phthalic acids, or fatty diacids, such as fatty acid dimer. Mention may also be made of the acids belonging to the preceding families carrying various electron-donating substituents (groups with heteroatom of the O or N type) or electron-withdrawing substituents (halogens, sulphonimides, nitro or sulphonato groups, or the like). Substituted aromatic acids are preferred.

Generally, the solvent is chosen in order advantageously to obtain a phase which is homogeneous under the temperature and pressure conditions at which the oxidation reaction is carried out. For this, it is advantageous for the solubility of the solvent in the hydrocarbon or the reaction medium to be at least greater than 2% by weight and for at least one homogeneous liquid phase comprising at least a portion of the hydrocarbons to be oxidized and a portion of the solvent to be formed.

Advantageously, the solvent is chosen from those which are not very soluble in water, that is to say which exhibit a solubility in water of less than 10% by weight at ambient temperature (10-30° C.).

However, it is possible, without departing from the scope of the invention, to use a solvent exhibiting a solubility in water greater than that indicated above if the partition coefficient of this compound between the organic phase or phases of the reaction medium, which are composed essentially of the hydrocarbon to be oxidized and the oxidation intermediates, and the nonorganic phase comprising the water formed during the oxidation reaction makes it possible to obtain a concentration of the solvent in the said aqueous phase of less than 10% by weight.

The oxidation is generally carried out in the presence of a catalyst. This catalyst advantageously comprises a metal component chosen from the group consisting of Cu, Ag, Au, Mg, Ca, Sr, Ba, Zn, Cd, Hg, Al, Sc, In, Tl, Y, Ga, Ti, Zr, Hf, Ge, Sn, Pb, V, Nb, Ta, Cr, Mo, W, Mn, Tc, Re, Fe, Ru, Os, Co, Rh, Ir, Ni, Pd, Pt, lanthanides, such as Ce, and the combinations of these.

These catalytic components are employed either in the form of compounds advantageously at least partially soluble in the liquid oxidation medium under the conditions for carrying out the oxidation reaction or supported on, absorbed by or bonded to an inert support, such as silica or alumina, for example.

The catalyst is preferably, in particular under the conditions for carrying out the oxidation reaction:
either soluble in the hydrocarbon to be oxidized,
or soluble in the solvent,
or soluble in the hydrocarbon/solvent mixture forming a homogeneous liquid phase under the conditions for carrying out the reaction.

According to a preferred embodiment of the invention, the catalyst used is soluble in one of these media at ambient temperature or the temperature for recycling these media to a further oxidation.

The term “soluble” is understood to mean that the catalyst is at least partially soluble in the medium under consideration.

In the case of heterogeneous catalysis, the catalytically active metal components are supported on or incorporated in a micro- or mesoporous inorganic matrix or in a polymer matrix or are in the form of organometallic complexes grafted to an organic or inorganic support. The term “incorporated” is understood to mean that the metal is a component of the support or that the operation is carried out with complexes sterically trapped in porous structures under the conditions of the oxidation.

In a preferred embodiment of the invention, the homogeneous or heterogeneous catalyst is composed of salts or of complexes of metals from groups IVb (Ti group), Vb (V group), VIb (Cr group), VIIb (Mn group), VIII (Fe or Co or Ni group), Ib (Cu group) and cerium, alone or as a mixture. The preferred components are in particular Mn and/or Co, in combination with one or more other metal components, such as, for example, Zr, Hf, Ce, Hf or Fe. The concentrations of metal in the liquid oxidation medium vary between 0.00001 and 5% (% by weight), preferably between 0.001% and 2%.

Furthermore, the concentration of solvent in the reaction medium is advantageously determined in order to have a molar ratio of the number of molecules of solvent to the catalytic metal atom number between 0.5 and 100 000, preferably between 1 and 5000.

The concentration of solvent in the liquid oxidation medium can vary within wide limits. Thus, it can be between 1 and 99% by weight, with respect to the total weight of the liquid medium; more advantageously, it can be between 2 and 50% by weight of the liquid medium.

It is also possible, without, however, departing from the scope of the invention, to use the solvent in combination with another compound which can in particular have the effect of improving the productive output and/or the selectivity of the oxidation reaction for adipic acid and in particular the dissolution of the oxygen.

Mention may in particular be made, as examples of such compounds, of nitrites, hydroxyimide compounds or halogenated compounds, more advantageously fluorinated compounds. Mention may be made, as compounds which are more particularly suitable, of nitriles, such as acetonitrile or benzonitrile, the imides belonging to the family disclosed in Patent Application EP 0 824 962, and more particularly N-hydroxysuccinimide (NHS) or N-hydroxyphthalimide (NHPI), or halogenated derivatives, such as dichloromethane or fluorinated compounds, such as:
fluorinated or perfluorinated cyclic or acyclic aliphatic hydrocarbons,
fluorinated aromatic hydrocarbons, such as perfluorotoluene, perfluoromethylcyclohexane, perfluoroheptane, perfluorooctane, perfluorononane, perfluorodecalin, perfluoromethyldecalin, α,α,α-trifluorotoluene or 1,3-bis(trifluoromethyl)benzene,
perfluorinated or fluorinated esters, such as perfluoro(alkyl octanoate)s or perfluoro(alkyl nonanoate)s,
fluorinated or perfluorinated ketones, such as perfluoroacetone,
fluorinated or perfluorinated alcohols, such as perfluorohexanol, perfluorooctanol, perfluorononanol, perfluorodecanol, perfluoro-t-butanol, perfluoroisopropanol or 1,1,1,3,3,3-hexafluoro-2-propanol,
fluorinated or perfluorinated nitriles, such as perfluoroacetonitrile,
fluorinated or perfluorinated acids, such as trifluoromethylbenzoic acids, pentafluorobenzoic acid, perfluorohexanoic acid, perfluoroheptanoic acid, perfluorooctanoic acid, perfluorononanoic acid or perfluoroadipic acid,
fluorinated or perfluorinated halides, such as perfluoroiodooctane or perfluorobromooctane,
fluorinated or perfluorinated amines, such as perfluorotripropylamine, perfluorotributylamine or perfluorotripentylamine.

The invention applies more particularly to the oxidation of cycloaliphatic compounds, such as cyclohexane or cyclododecane, to give the corresponding linear diacids, adipic acid or dodecanedioic acid.

According to a preferred embodiment of the invention, the invention relates to the direct oxidation of cyclohexane to give adipic acid by oxygen or a gas comprising oxygen in a liquid medium and in the presence of a manganese catalyst or a manganese/cobalt combination.

The oxidation reaction is carried out at a temperature of between 50° C. and 200° C., preferably between 70° C. and 180° C. It can be carried out at atmospheric pressure. However, it is generally carried out under pressure in order to keep the components of the reaction medium in the liquid form. The pressure can be between 10 kPa (0.1 bar) and 20 000 kPa (200 bar), preferably between 100 kPa (1 bar) and 10 000 kPa (100 bar).

The oxygen used can be in the pure form or as a mixture with an inert gas, such as nitrogen or helium. It is also possible to use air more or less enriched in oxygen. The amount of oxygen fed to the medium is advantageously between 1 and 1000 mol per mole of compounds to be oxidized.

The oxidation process can be carried out continuously or according to a batchwise process. Advantageously, the liquid reaction medium exiting from the reactor is treated according to known processes which make it possible, on the one hand, to separate and recover the diacids produced and, on the other hand, to recycle the nonoxidized or partially oxidized organic compounds, such as cyclohexane, cyclohexanol and/or cyclohexanone. It is advantageous to also employ a compound which initiates the oxidation reaction, such as, for example, a ketone, an alcohol, an aldehyde or a hydroperoxide. Cyclohexanone, cyclohexanol and cyclohexyl hydroperoxide, which are reaction intermediates in the case of the oxidation of cyclohexane, are very particularly indicated. Generally, the initiator represents from 0.01% to 20% by weight of the weight of the reaction mixture employed, without these proportions having a critical value. The initiator is useful in particular when starting the oxidation. It can be introduced from the beginning of the reaction.

The oxidation can also be carried out in the presence of water introduced from the initial stage of the process.

As indicated above, the reaction mixture resulting from the oxidation is subjected to various operations for separating some of its constituents in order, for example, to make it possible to recycle them to the oxidation and to make it possible to recover the acids produced.

According to a first embodiment of the invention, the medium exiting from the oxidation reactor is subjected directly to a second oxidation stage in the presence of a homogeneous or heterogeneous metal catalyst. The temperature and pressure conditions can be identical to or different from the conditions used in the stage for oxidation of the hydrocarbon. The oxidizing agent used can be oxygen, a gas comprising oxygen, aqueous hydrogen peroxide solution, ozone, an organic hydroperoxide or the like, for example. In this oxidation stage, the α,ω-hydroxycarboxylic compound formed, such as hydroxycaproic acid in the case of the oxidation of cyclohexane, is converted to dicarboxylic acid. As indicated above, this stage is carried out either in the oxidation reactor or in one or more additional reactors.

The catalyst is advantageously a homogeneous catalyst composed of at least one compound of a metal chosen from the group consisting of Cu, Ag, Au, Mg, Ca, Sr, Ba, Zn, Cd, Hg, Al, Sc, In, Tl, Y, Ga, Ti, Zr, Hf, Ge, Sn, Pb, V, Nb, Ta, Cr, Mo, W, Mn, Tc, Re, Fe, Ru, Os, Co, Rh, Ir, Ni, Pd, Pt, lanthanides, such as Ce, and the combinations of these.

It is also possible to use a heterogeneous catalyst comprising, as catalytic phase, one of the above metal compounds.

On conclusion of this stage, the reaction mixture is cooled and separated by settling into at least two liquid phases: one or more organic phases essentially comprising unreacted hydrocarbon, optionally the solvent and certain oxidation intermediates, such as alcohols and ketones, and an aqueous phase comprising the diacids formed during the oxidation of the hydrocarbon and during the stage of conversion of the α,ω-hydroxycarboxylic compounds.

Advantageously, the organic phase is washed several times with water or an acidic aqueous solution in order to extract the maximum amount of dicarboxylic acids.

The organic phase, which comprises the nonoxidized hydrocarbon (cyclohexane) and certain intermediate oxidation compounds, such as cyclohexanone or cyclohexanol, is recycled, advantageously, to the stage of oxidation of the hydrocarbon.

In the case where the acid solvent is a solvent possessing a lipophilic nature, this solvent is present in the organic phase as it is insoluble in water. For this reason, it is recycled to the oxidation stage with the nonoxidized cyclohexane. This recycling of solvent occurs in particular when the solvent is chosen from substituted or unsubstituted aromatic acids, such as tert-butylbenzoic acid.

The diacids formed, in particular adipic acid, are recovered from the aqueous phase, for example by crystallization.

The acids thus recovered are advantageously purified according to the standard techniques described in numerous documents. Among the purification methods, purification by crystallization from various solvents, such as water, aqueous acetic acid solution or alcohols, is preferred. Purification methods are disclosed in particular in French Patents Nos. 2 749 299 and 2 749 300.

Likewise, if the catalyst for the oxidation of the hydrocarbon is not completely recycled with the organic phase and is partly or completely extracted with the aqueous phase, it will advantageously be extracted from the aqueous phase by various techniques, such as liquid/liquid extraction, electrodialysis or treatment on ion-exchange resins, for example.

In a second embodiment of the invention, the stage of oxidation of the α,ω-hydroxycarboxylic compounds, such as 6-hydroxycaproic acid, is carried out on the aqueous phase recovered after the stage of cooling the oxidation reaction medium and separating it by settling and/or on the aqueous phase from washing the organic phase, and also on the aqueous mother liquors recovered during the crystallization of the dicarboxylic acid.

In this second embodiment, the oxidation of the α,ω-hydroxycarboxylic compounds is carried out in the presence or the absence of catalyst by oxygen or a gas comprising oxygen, such as air, for example. It is also possible to use other oxidizing agents, such as nitric acid, aqueous hydrogen peroxide solution or ozone. The oxidation reaction is carried out at a temperature of between 50° C. and 150° C. and under an oxygen pressure of between 0.1 and 30 bar of oxygen partial pressure.

Advantageously, the catalyst used is a heterogeneous catalyst, for example a supported catalyst comprising, as catalytically active metal entity, a compound or a mixture of compounds of metal components chosen from the group consisting of Au, Pt, Ru, Cr, Ti, V, Mn, Fe, Co, Zn, Mo, Rh, Pd, Ag, W, Re, Os and Bi. Mention may be made, as catalyst which is particularly suitable for the invention, of catalysts based on platinum supported on charcoal, alumina or titanium oxide or a catalyst based on platinum and bismuth supported on charcoal.

This oxidation operation can be carried out on all the aqueous phases recovered during the extraction and the purification of the dicarboxylic acid, in particular on the aqueous crystallization mother liquors. It can also be carried out simultaneously with the separation by settling of the aqueous and organic phases.

After oxidation, the aqueous medium recovered is treated as above to extract the diacids, in particular adipic acid.

Advantageously, the process of the invention can comprise a stage of hydrolysis of the esters formed in the oxidation stage. Such a hydrolysis stage is disclosed in French Patent 2 846 651, for example.

This hydrolysis stage is advantageously and preferably carried out on the organic phase recovered after the cooling and separating by settling/washing stage.

The process of the invention makes it possible to manufacture a diacid by oxidation of a cyclic hydrocarbon by oxygen or a gas comprising oxygen, with recycling of the nonoxidized hydrocarbon, without accumulation of the various by-products formed in the oxidation stage. Furthermore, the diacid or diacids recovered can be easily purified as they are not contaminated by certain by-products from the reaction for the oxidation of the hydrocarbon.

Other advantages and details of the invention will become more clearly apparent in the light of the examples, given solely by way of illustration.

### EXAMPLE 1

### Comparative

600 g of an aqueous solution obtained by separation of the reaction medium originating from the oxidation of cyclohexane by air in the presence of tert-butylbenzoic acid and of a catalyst based on manganese and cobalt as disclosed in French Patent No. 2 828 194 comprise in particular:
adipic acid: 30%
succinic acid: 2.35%
glutaric acid: 5.60%
6-hydroxycaproic acid: 4.46%

The aqueous solution obtained is cooled in order to obtain crystalline adipic acid. The solid obtained after filtration is washed with water and then taken up in 300 ml of water with heating.

The new solution is cooled in order to make possible the crystallization of the adipic acid. This operation is repeated once.

The hydroxycaproic acid is quantitatively determined in the adipic acid collected after each crystallization:
1st crystallization: 1986 ppm
2nd crystallization: 73 ppm
3rd crystallization: 22 ppm

This test shows that it is necessary to carry out at least three successive crystallizations of the adipic acid in order to obtain a low concentration of hydroxycaproic acid in the adipic acid corresponding to the required specifications.

### EXAMPLE 2

580 g of a reaction medium obtained during the oxidation of cyclohexane by air in the presence of tert-butylbenzoic acid and of a catalyst based on manganese and cobalt as disclosed in French Patent No. 2 828 194 are washed with 250 ml of water in order to extract the various water-soluble compounds, in particular the acids formed and 6-hydroxycaproic acid.

The resulting aqueous phase comprises in particular 1% by weight of formic acid, 0.7% by weight of succinic acid, 3.4% by weight of glutaric acid, 7.3% by weight of adipic acid and 1.4% by weight of 6-hydroxycaproic acid (HOCap). 3.65 g of this aqueous phase are charged to an autoclave agitated by shaking in the presence of Pt supported on powdered charcoal sold by Engelhardt (HOCap/Pt molar ratio=15). The reaction takes place under an air pressure of 25 bar at 90° C. for 3 hours. After analysis by chromatography, the test results in a conversion of the 6-hydroxycaproic acid of 100%, a conversion of the formic acid of 100% and a true yield of adipic acid of 80%, with respect to the 6-hydroxycaproic acid involved. The mixture obtained is treated by conventional methods for the crystallization of adipic acid. The content of 6-hydroxycaproic acid (HOCap) in the adipic acid after a first crystallization is less than 2 ppm.

### EXAMPLE 3

Example 2 is repeated but while replacing air with H₂O₂ and the supported platinum catalyst with 13 mg of tungstic acid in the stage of oxidation of the 6-hydroxycaproic acid.

After heating at 20° C. for 4 hours, 20.4% of the hydroxycaproic acid is converted to adipic acid.

### EXAMPLE 4

Example 2 is repeated but while replacing, in the stage of oxidation of the 6-hydroxycaproic acid, air with a 60% by weight nitric acid solution and while using, as catalyst, a composition comprising 6000 ppm by weight, expressed as copper, of copper nitrate (Cu(NO₃)₂·3H₂O) and 300 ppm, expressed as vanadium, of VO₃NH₄.

The reaction is carried out at 70° C. for 3 hours. The 6-hydroxycaproic acid is completely converted. The adipic acid yield is 68% with respect to the 6-hydroxycaproic acid involved.

### EXAMPLE 5

Example 2 is repeated but while replacing the platinum-on-charcoal catalyst with palladium acetate added at a concentration of 10% by weight.

The degree of conversion of the 6-hydroxycaproic acid is 100%. The adipic acid yield is 63% with respect to the 6-hydroxycaproic acid involved.

### EXAMPLE 6

Example 2 is repeated but while replacing the platinum-on-charcoal catalyst with a supported catalyst composed of alumina as support and an Ag/Pd combination as supported catalytic phase. The concentration of the catalytic phase, expressed as weight of metal, is 10% by weight with respect to the alumina support.

The degree of conversion of the 6-hydroxycaproic acid is 57% and the adipic acid yield is 59% with respect to the 6-hydroxycaproic acid converted.

### EXAMPLE 7

Example 2 is repeated but while replacing the platinum-on-charcoal catalyst with a supported catalyst composed of active charcoal as support and an Ru/Fe combination as supported catalytic phase. The concentration of the catalytic phase, expressed as weight of metal, is 10% by weight with respect to the active charcoal support.

The degree of conversion of the 6-hydroxycaproic acid is 86% and the adipic acid yield is 46% with respect to the 6-hydroxycaproic acid converted.

### EXAMPLE 8

Example 2 is repeated but while replacing the platinum-on-charcoal catalyst with a supported catalyst composed of graphite as support and a Pt/Bi combination as supported catalytic phase. The concentration of the catalytic phase, expressed as weight of metal, is 10% by weight with respect to the graphite support.

The degree of conversion of the 6-hydroxycaproic acid is 96% and the adipic acid yield is 81% with respect to the 6-hydroxycaproic acid converted.

### EXAMPLE 9

Example 2 is repeated but while replacing the platinum-on-charcoal catalyst with a supported catalyst composed of alumina as support and a Pt/Bi combination as supported catalytic phase. The concentration of the catalytic phase, expressed as weight of metal, is 10% by weight with respect to the alumina support.

The degree of conversion of the 6-hydroxycaproic acid is 82% and the adipic acid yield is 69% with respect to the 6-hydroxycaproic acid converted.

### EXAMPLE 10

Example 2 is repeated but while replacing the platinum-on-charcoal catalyst with a supported catalyst composed of titanium oxide as support and platinum as supported catalytic phase. The concentration of the catalytic phase, expressed as weight of metal, is 10% by weight with respect to the titanium oxide support.

The degree of conversion of the 6-hydroxycaproic acid is 100% and the adipic acid yield is 69% with respect to the 6-hydroxycaproic acid converted.

1.-16. (canceled)

17. A process for the production of dicarboxylic acids by oxidation of a hydrocarbon with oxygen or a gas containing oxygen in the presence of a solvent, which process comprises:
oxidizing the hydrocarbon,
extracting the diacids formed from the reaction medium by liquid/liquid extraction with water or an aqueous solution of acids as extraction solvent,
recovering the diacids formed by crystallization from the aqueous phase recovered on conclusion of said liquid/liquid extraction,
recycling, to the oxidation stage, the organic phase recovered on conclusion of said oxidation stage, and further comprising
a stage of conversion, removal or extraction of the α,ω-hydroxycarboxylic compounds formed in the oxidation stage and which entails oxidizing said hydroxycarboxylic compounds to provide said dicarboxylic acids.

18. The process as defined by claim 17, comprising one or more stages of crystallization of the diacids from an aqueous phase.

19. The process as defined by claim 17, wherein the oxidation of said hydroxycarboxylic compounds is carried out on the reaction medium on conclusion of the oxidation reaction.

20. The process as defined by claim 19, wherein the oxidation of said α,ω-hydroxycarboxylic compounds is carried out by addition of a catalyst to the oxidation reactor at the end of oxidation of the hydrocarbon.

21. The process as defined by claim 19, wherein the oxidation of said α,ω-hydroxycarboxylic compounds is carried out in one or more additional oxidation reactors.

22. The process as defined by claim 19, wherein, the catalyst is a catalyst which is soluble in the reaction medium.

23. The process as defined by claim 22, wherein the acids formed are extracted by liquid/liquid extraction.

24. The process as defined by claim 23, wherein the extraction solvent is water.

25. The process as defined by claim 17, wherein the oxidation of the α,ω-hydroxycarboxylic compounds is carried out on the aqueous phase or phases recovered after the stage of liquid/liquid extraction of the diacids and/or the aqueous mother liquors from crystallization of the diacids.

26. The process as defined by claim 25, wherein the oxidation is carried out at a temperature of from 50° C. to 150° C. and an oxygen partial pressure of from 0.1 to 30 bar.

27. The process as defined by claim 25, wherein the oxidation is carried out in the presence of a metal catalyst.

28. The process as defined by claim 27, wherein the metal catalyst is selected from the group consisting of Cu, Ag, Au, Mg, Ca, Sr, Ba, Zn, Cd, Hg, Al, Sc, In, Tl, Y, Ga, Ti, Zr, Hf, Ge, Sn, Pb, V, Nb, Ta, Cr, Mo, W, Mn, Tc, Re, Fe, Ru, Os, Co, Rh, Ir, Ni, Pd, Pt, lanthanides, Ce, and combinations thereof.

29. The process as defined by claim 27, wherein the catalyst is a supported catalyst comprising an active phase of one or more components selected from the group consisting of Cu, Ag, Au, Mg, Ca, Sr, Ba, Zn, Cd, Hg, Al, Sc, In, Tl, Y, Ga, Ti, Zr, Hf, Ge, Sn, Pb, V, Nb, Ta, Cr, Mo, W, Mn, Tc, Re, Fe, Ru, Os, Co, Rh, Ir, Ni, Pd, Pt, lanthanides, Ce, and combinations thereof, and a support selected from the group consisting of alumina, silica, zeolites and charcoals.

30. The process as defined by claim 28, wherein said catalyst comprises precious metals selected from the group consisting of gold, platinum, palladium, ruthenium or silver.

31. The process as defined by claim 17, wherein said hydrocarbon is selected from the group consisting of cyclohexane and cyclododecane.

32. The process as defined by claim 17, said solvent comprising a lipophilic acid.
